(19) United States
(12) Patent Application Publication
(10) Pub. No.: US 2024/0075304 A1
(43) Pub. Date: Mar. 7, 2024

(54) IN-BAND TELEMETRY FOR DEVICE LONGEVITY

(71) Applicant: Medtronic, Inc., Minneapolis, MN (US)

(72) Inventors: Ashutosh Mehra, St. Paul, MN (US); Nathan A. Torgerson, Andover, MN (US); Venkat R. Gaddam, Plymouth, MN (US); Arthur K. Lai, Minnetonka, MN (US); Bernard P. Bechara, Minneapolis, MN (US); Joel A. Anderson, Brooklyn Park, MN (US); Brent P. Johnson, Shoreview, MN (US); Trevor D. Webster, Minneapolis, MN (US); Mandla T. Shongwe, Brooklyn Park, MN (US); Cesar G. Moran, St. Paul, MN (US); Charles M. Nowell, Jr., Longwood, FL (US)

(21) Appl. No.: 18/461,957

(22) Filed: Sep. 6, 2023

Related U.S. Application Data
(60) Provisional application No. 63/374,698, filed on Sep. 6, 2022.

(51) Int. Cl. A61N 1/372; A61N 1/378; G08C 17/02

(52) U.S. Cl. CPC A61N 1/37223; A61N 1/3787; G08C 17/02

(57) ABSTRACT

A medical device includes wake circuitry and telemetry circuitry. The wake circuitry is configured to receive a first set of data from a device associated with the medical device, where the first set of data is received at a frequency band. The wake circuitry is configured to output a set of pulses based on the first set of data. The wake circuitry is configured to detect a data pattern using the set of pulses. The wake circuitry is configured to output an activation signal in response to a determination that the data pattern satisfies a data pattern requirement. The telemetry circuitry is configured to output a second set of data in response to receiving the activation signal. The second set of data is transmitted at the frequency band. The telemetry circuitry is configured to establish a communication session with the device using the second set of data.

This application claims the benefit of U.S. Provisional Application Ser. No. 63/374,698, filed Sep. 6, 2022, the entire content of which is incorporated herein by reference.

### TECHNICAL FIELD

The disclosure relates to medical devices and, more particularly, medical devices that deliver therapy to a patient.

### BACKGROUND

Medical devices may be external or implanted, and may be used to sense neural signals (e.g., central and peripheral nerves) and/or deliver electrical stimulation therapy to various tissue sites of a patient to treat a variety of symptoms or conditions such as, for example, one or more of chronic pain, tremor, Parkinson's disease, other movement disorders, epilepsy, urinary or fecal incontinence, sexual dysfunction, obesity, gastroparesis, sleep apnea, neural control of prosthetic devices, or stimulation to provide peripheral sensation. A medical device delivers electrical stimulation therapy via one or more leads that include electrodes located proximate to target locations associated with the brain, the spinal cord, pelvic nerves, peripheral nerves, or the gastrointestinal tract of a patient. For bipolar stimulation, the electrodes used for stimulation may be on one or more leads. For unipolar stimulation, the electrodes may include one or more leads and an electrode on a stimulator housing located remotely from the target site (e.g., near clavicle). It may be possible to use leadless stimulation using electrodes mounted on the stimulator housing. Hence, electrical stimulation is used in different therapeutic applications, such as deep brain stimulation (DBS), spinal cord stimulation (SCS), pelvic stimulation, gastric stimulation, or peripheral nerve field stimulation (PNFS).

A clinician may select values for a number of programmable parameters in order to define the electrical stimulation therapy to be delivered by the implantable stimulator to a patient. For example, the clinician may select one or more electrodes for delivery of the stimulation, a polarity of each selected electrode, a voltage or current pulse amplitude, a pulse width, and a pulse rate as stimulation parameters. A set of parameters, such as a set including electrode combination, electrode polarity, amplitude, pulse width, and pulse rate, may be referred to as a program in the sense that they define the electrical stimulation therapy to be delivered to the patient.

### SUMMARY

This disclosure describes example techniques for improving energy efficiency of telemetry circuitry of a medical device (e.g., an implantable medical device). The medical device may include wake circuitry configured to output a set of pulses (e.g., voltage signals) based on a first set of data from a device associated with the medical device (e.g., an external device). Responsive to the set of pulses satisfying a data pattern requirement, the wake circuitry may output an activation signal that causes the telemetry circuitry to establish a communication session with the device using a second set of data.

The first set of data and the second set of data may be transmitted at the same frequency band. That is, the signal the wake circuitry receives from the device associated with the medical device may be at the same frequency band as the signal the telemetry circuitry sends to the device to establish a communication session. This configuration may be referred to as an in-band sting mechanism. An in-band sting mechanism may advantageously allow for reduced design complexity. For example, an in-band sting mechanism in accordance with techniques of this disclosure may allow for removal of coils and decrease the variety of components (e.g., the number of different types of antennas) for the medical device and/or the device. Additionally, the techniques may improve responsiveness of communication between the device and the medical device while minimizing energy drain of the medical device.

In some examples, a medical device configured to provide a therapy to a patient comprises: wake circuitry configured to: receive a first set of data from a device associated with the medical device, wherein the first set of data is received at a frequency band; output a set of pulses based on the first set of data; detect a data pattern using the set of pulses; and responsive to a determination that the data pattern satisfies a data pattern requirement, output an activation signal; and the telemetry circuitry configured to: responsive to receiving the activation signal, output a second set of data, wherein the second set of data is transmitted at the frequency band; and establish a communication session with the device using the second set of data.

In some examples, a method comprises: receiving, by wake circuitry of a medical device configured to provide a therapy to a patient, a first set of data from a device associated with the medical device, wherein the first set of data is received at a frequency band; outputting, by the wake circuitry, a set of pulses based on the first set of data; detecting, by the wake circuitry, a data pattern using the set of pulses; responsive to a determination that the data pattern satisfies a data pattern requirement, outputting, by the wake circuitry, an activation signal; responsive to receiving, by telemetry circuitry of the medical device, the activation signal, outputting, by the telemetry circuitry, a second set of data, wherein the second set of data is transmitted at the frequency band; and establishing, by the telemetry circuitry, a communication session with the device using the second set of data.

In some examples, a system comprises: a device associated with a medical device configured to provide a therapy to a patient, wherein the device comprises one or more of an additional medical device, a clinician programmer, a patient programmer, a recharger device, or a mobile device; and the medical device comprising: wake circuitry configured to: receive a first set of data from the device, wherein the first set of data is received at a frequency band; output a set of pulses based on the first set of data; detect a data pattern using the set of pulses; and responsive to a determination that the data pattern satisfies a data pattern requirement, output an activation signal; and the telemetry circuitry configured to: responsive to receiving the activation signal, output a second set of data, wherein the second set of data is transmitted at the frequency band; and establish a communication session with the device using the second set of data.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes an implantable medical device (IMD) according to an example of the techniques of the disclosure.

FIG. 2 is a conceptual diagram illustrating an example system that includes an IMD according to an example of the techniques of the disclosure.

FIG. 3 is a block diagram of an example IMD according to an example of the techniques of the disclosure.

FIG. 4 is a block diagram of an example IMD according to an example of the techniques of the disclosure.

FIG. 5 is a block diagram of an example IMD according to an example of the techniques of the disclosure.

FIG. 6 is a flowchart illustrating an example operation of an example IMD according to an example of the techniques of the disclosure.

### DETAILED DESCRIPTION

A system may be configured to deliver electrical stimulation therapy (e.g., neuromodulation such as deep brain stimulation (DBS), spinal cord stimulation (SCS), sacral nerve stimulation (SNS), peripheral nerve stimulation therapy, etc.). In some examples, the system may be configured to adjust program parameters for a medical device (e.g., an implantable medical device or IMD) using a device associated with the medical device, such as, an additional medical device, a clinician programmer, a patient programmer, a recharger device, or a mobile device. Electrical stimulation therapy may be delivered via multiple electrodes of one or more leads (e.g., cylindrical or paddle leads) implanted to provide stimulation in the brain, in the spinal cord, in the sacral nerve, or the percutaneous tibial nerve stimulation of a patient. In some examples, electrical stimulation therapy may be delivered via a leadless device. Electrical stimulation therapy may be adaptively adjusted for a patient using at least one program parameter.

A medical device, such as an IMD, of a system may include telemetry circuitry to communicate with other devices at a distance. The telemetry circuitry may use Bluetooth™ (ISM frequency bands from 2.402 GHz to 2.48 GHz) or other wireless technology protocols (e.g., radiofrequency (RF) protocols) to implement communication with devices associated with the medical device (e.g., proprietary external programmers, commercial off-the-shelf instruments such as cellphones and tablets, etc.). Access to external instruments may help to enable recharge and product longevity, and may help to prevent high risk procedures like replacement surgeries for implantable devices.

In some examples, an IMD may be a peripheral device that transmits a set of data, such as a set of advertisements, to pair, bond and connect to companion devices (e.g., devices associated with the IMD). Connecting to a companion device may require the telemetry circuit to be active and in turn draining a higher rate of current. In general, current drain may vary based on the rate of data transmission, A faster data transmission rate may result in higher current drain and hence lower battery life. Thus, the impact of high frequency protocols may be high on device longevity and is directly proportional to connection and/or data transmission intervals. On the other hand, a faster data transmission rate may result in a faster response time. As such, faster data transmission may lead to faster connection and reduced latency, but may also lead to higher current drain and reduced device longevity.

In another example, the IMD may operate as a central by scanning for peripheral devices. Once the telemetry circuitry locates a relevant companion radio, the IMD may switch to the role of a peripheral device and transmit advertisements to connect. Although this configuration reduces transmission current drain, the telemetry circuitry may need to be active for a longer duration, which may offset any reduction in transmission current drain. Also, the time lag between changing roles may increase latency.

The energy to either maintain a distance telemetry session or make a medical device immediately responsive for the user can consume a significant proportion of the energy from the medical device, which may reduce longevity of the medical device. The energy consumption of the medical device may necessitate replacement of the medical device or battery, which may involve high risk procedures like replacement surgeries for the medical device.

A system according to techniques described herein may include telemetry circuitry configured to transition from an inactive state (e.g., a “deep sleep” mode) to an active state in response to an activation signal (e.g., a “sting”) from wake circuitry of the system. When active, the telemetry circuitry may output a set of data (e.g., a set of advertisements) for connecting to a device associated with the medical device. The wake circuitry may output the activation signal to activate the telemetry circuitry in response to receiving a signal, such as a set of data, from a device associated with the medical device. In some examples, the wake circuitry may determine whether a received signal is a signal from a device associated with the medical device when a data pattern requirement is satisfied.

When the telemetry circuitry is inactive, the functionality of the telemetry circuitry may be limited to reduce energy consumption. Thus, the telemetry circuitry may consume less energy when the telemetry circuitry is inactive compared to when the telemetry circuitry is active. When the telemetry circuitry is active, the telemetry circuitry may be configured to establish a communication session with the device associated with the medical device. For example, responsive to activation, the telemetry circuitry may establish a communication session using a set of data that are transmitted at the same frequency band as the set of advertisements from the device associated with the medical device. Thus, the techniques of this disclosure may advantageously allow the telemetry circuitry to conserve energy (e.g., by being inactive) when no signal is detected from a device associated with the medical device and allow the telemetry circuitry to activate and establish a communication session when a signal is detected from a device associated with the medical device.

Implantable medical devices (IMDs) with limited battery capacity (e.g., a primary battery and/or a rechargeable battery) may benefit from techniques described herein. The IMD may communicate via a wireless protocol (e.g., Bluetooth™ or another protocol) to a number of different instruments, such as, for example, an additional medical device, a patient programmer, a clinician programmer, a recharger device (also referred to herein as simply “recharger”), a programming fob, or another device. In some examples, an IMD may consume a significant amount of energy stored in the battery to establish connections and would benefit from techniques that reduce the amount of energy consumed by the telemetry circuitry while idle (because, e.g., there are no devices associated with the medical device nearby and/or attempting to communicate with the medical device). Saving energy may help to optimize telemetry performance and/or efficiency, which can help make the medical device (e.g., an implantable medical device) last longer. This may in turn reduce the number of replacement surgeries that are necessary, improving patient outcomes.

As used herein, the data of the set of data may refer to any modulated RF signal. The modulated RF signal may (or may not) include encoded data and/or data packets. For example, on/off keying of an RF signal may constitute data for purposes of this disclosure. In any case, the RF signal may be at any frequency band appropriate for communication between devices. For example, the RF signal may be at a frequency band from 2.402 GHz to 2.48 GHz, any other frequency bands in the protocols discussed herein, etc.

Additionally, this disclosure primarily describes examples wherein the set of data for establishing communication between devices of a medical system includes a set of advertisements (e.g., a set of Bluetooth Low Energy (BLE) advertisements) that include information for connecting devices. However, it should be understood that the sets of advertisement described below are provided as an example for purposes of explanation only, and that a set of data is not limited to a set of advertisements. Thus, examples other than a set of advertisements are contemplated by this disclosure.

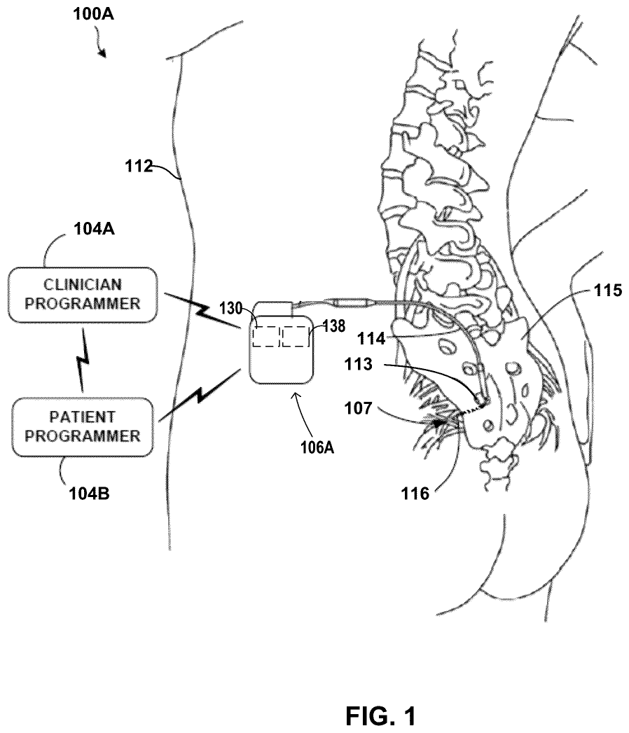

FIG. 1 is conceptual diagram illustrating an example system 100A configured to deliver stimulation therapy to a patient 112 using one or more sequences of different pulse trains as described herein. Patient 112 is ordinarily a human patient. In some cases, however, system 100A may be applied to other mammalian or non-mammalian, non-human patients. In some examples, patient 112 experiences bladder dysfunction, such as, for example, improper functioning of a bladder, a urinary sphincter, or a urinary tract, and may include an overactive bladder, urgency, urinary incontinence, urgency frequency, urinary retention, or combinations thereof. In some examples, the systems and techniques described herein may also be applied to the delivery of stimulation to nerves and/or muscles in order to treat bowel disfunction such as fecal incontinence or other disorders.

System 100A may be configured to deliver stimulation therapy using stimulation pulses. System 100A may include an IMD 106A as well as devices associated with IMD 106A, such as a clinician programmer 104A and/or a patient programmer 104B (collectively, “device 104”). IMD 106A may provide electrical stimulation therapy to a target tissue site 107 located proximate a sacral nerve, a pudendal nerve, a hypogastric nerve, a pelvic nerve, tibial nerve, saphenous nerve, or another nerve associated with the bladder of patient 112 by generating a programmable electrical stimulation signal (e.g., in the form of electrical pulses) and delivering the electrical stimulation signal to target tissue site 107 via one or more leads 114. In some examples, lead 114 includes one or more stimulation electrodes 116 proximate to target tissue site 107 such that the electrical stimulation is delivered from IMD 106A to target tissue site 107 via the stimulation electrodes. The electrical stimulation therapy may be used to treat bladder dysfunction of patient 112.

In general, the sacral nerves include five sacral nerves that emerge from the sacrum. In some examples, the sacral vertebrae (S1-S5) may be used to number the sacral nerves. The sacral nerves contribute to the sacral plexus (a network of intersecting nerves that innervates the posterior thigh, part of the lower leg, the foot, and part of the pelvis) and the coccygeal plexus (a network of intersecting nerves near the coccyx bone, e.g., the tailbone, that innervates the skin of the coccyx bone and around the anus). In general, the pudendal nerve is a somatic nerve in the pelvic region, which is a large branch of the sacral plexus. The pudendal nerve innervates the external genitalia, the urinary sphincters, and the anal sphincters. The hypogastric nerves and pelvic nerves innervate the detrusor muscle of the bladder. Stretch receptors of the bladder may send signals to the brain of patient 112 via afferent sensory fibers in sacral nerves that may result in increased activity of the detrusor muscle of the bladder.

As illustrated in the example of FIG. 1, electrodes 116 of lead 114 are implanted proximate to target tissue site 107. In the example shown in FIG. 1, target tissue site 107 is proximate the S3 sacral nerve of patient 112. In this example, in order to implant electrodes 116 of lead 114 proximate to the S3 sacral nerve, lead 114 may be introduced into the S3 sacral foramen 113 of sacrum 115 to access the S3 sacral nerve. For some patients, stimulation of the S3 sacral nerve may be effective in treating bladder dysfunction of patient 112. In other examples, electrodes 116 may be implanted proximate to a different target tissue site 107, such as a target tissue site 107 proximate to a different sacral nerve, a pudendal nerve, a hypogastric nerve, a pelvic nerve, an afferent nerve, or another nerve associated with the bladder of patient 112 to treat the bladder dysfunction of patient 112. Electrodes 116 of lead 114 may include multiple electrodes, such as four or more electrodes.

Although FIG. 1 illustrates one lead 114, in some examples, IMD 106A may be coupled to two or more leads, e.g., to facilitate bilateral (e.g., stimulation to sacral nerves on either side of the spinal cord) or multi-lateral stimulation (e.g., stimulation to multiple sacral nerves on the same side of the spinal cord). In some examples, lead 114 may sense one or more physiological parameters (e.g., nerve signals, EMG, or the like) of patient 112 using electrodes 116. Alternatively, lead 114 may stimulate a target tissue site 107 via electrodes 116. In other examples, lead 114 may stimulate and sense using electrodes 116. In some examples, lead 114 includes a lead body, and a proximal end of lead 114 may be electrically coupled to IMD 106A via one or more conductors extending substantially through the lead body between the one or more stimulation electrodes carried by lead 114 and IMD 106A. In the case of an external device, the proximal end may be configured to couple to an external medical device, such as trial neurostimulator.

In the example shown in FIG. 1, lead 114 is cylindrical. One or more electrodes 116 of lead 114 may be ring electrodes, segmented electrodes, or partial ring electrodes. Segmented or partial ring electrodes each extend along an arc less than 360 degrees (e.g., 70-120 degrees) around the outer perimeter of the lead 114, where in some example, multiple electrode segments are disposed around the perimeter of lead 114 at the same axial position of lead 114. In some examples, segmented electrodes may be useful for targeting different fibers of the same or different nerves to generate different physiological effects or for delivering relatively higher frequency stimulation (e.g., about 66 Hertz) and relatively lower frequency stimulation (e.g., about 15 Hertz) to activate both fast twitch muscles and slow twitch muscles substantially simultaneously or at alternating time slots. In some examples, different pulse width values may be selected in order to selectively stimulate certain nerve fibers while avoiding other nerve fibers (e.g., fibers having different diameters). In some examples, lead 114 may include a paddle-shaped (e.g., a “paddle” lead) portion with a flat or curved surface.

In some examples, one or more electrodes 116 of lead 114 may be cuff electrodes that are configured to extend at least partially around a nerve (e.g., extend axially around an outer surface of a nerve). In some cases, delivering stimulation via one or more cuff electrodes and/or segmented electrodes may help achieve a more uniform electrical field or activation field distribution relative to the nerve in some examples, which may help minimize discomfort to patient 112 that results from the delivery of electrical stimulation. An electrical field represents the areas of a patient anatomical region that are covered by an electrical field during delivery of electrical stimulation to tissue within patient 112. The electrical field may define the volume of tissue that is affected when electrodes 116 of lead 114, or any other electrodes carried by IMD 106A (e.g., one or more electrodes carried by the housing of IMD 106A), are activated. An activation field represents the neurons that will be activated by the electrical field in the neural tissue proximate to the activated electrodes.

The illustrated numbers and configurations of lead 114 and electrodes 116 carried by lead 114 are merely one example. Different configurations, e.g., different quantities and/or positions of leads and electrodes, are possible. In other examples, IMD 106A may be coupled to additional leads or lead segments having one or more electrodes positioned at different locations in the pelvic region of patient 112. In some examples, lead 114 may have one electrode, two electrodes, three electrodes, four electrodes, or eight electrodes. In other examples, lead 114 may have a combination of ring electrodes and segmented electrodes.

IMD 106A may be surgically implanted in patient 112 at any suitable location within patient 112, such as within in an abdomen of patient 112. In some examples, the implantation site may be a subcutaneous location in the upper buttocks, side of the lower abdomen, or the side of the lower back. IMD 106A has a biocompatible outer housing, which may be formed from titanium, stainless steel, a liquid crystal polymer, or the like. In some examples, such as with a leadless stimulator, the outer housing may have an electrically insulative coating (e.g., parlyene). In some examples, electrical conductors disposed within the lead body of lead 114 electrically connect electrodes to an electrical stimulation delivery module within IMD 106A. In other examples, system 100A may include a leadless electrical stimulator, such as a microstimulator (e.g., a capsule shaped microstimulator), where the leadless electrical stimulator delivers electrical stimulation to target tissue site 107.

System 100A may include device 104, such as clinician programmer 104A and/or patient programmer 104B. In some examples, device 104 may be a wearable communication device integrated into a remote, key fob, or a wristwatch. In other examples, device 104 may be a handheld computing device, computer workstation, smartphone, personal computer, or networked computing device. Device 104 may be a proprietary device or computing device configured to execute different programs, one or more of which may include an application that, when executed, causes device 104 to function as described herein. Device 104 may include respective user interfaces that receive input from a user (e.g., a clinician or patient 112, respectively). The user interfaces may include components for interaction with a user, such as a keypad and a display. In some examples, the display may be a cathode ray tube (CRT) display, a liquid crystal display (LCD), or light emitting diode (LED) display and the keypad may take the form of an alphanumeric keypad or a reduced set of keys associated with particular functions. Device 104 can, additionally or alternatively, include a peripheral pointing device, e.g., a mouse, via which a user may interact with the user interface. In some examples, the displays may include a touch screen display, and a user may interact with device 104 via the touch screens of the displays. In some examples, the user may also interact with device 104 and/or IMD 106A remotely via a networked computing device.

Clinician programmer 104A facilitates interaction of a clinician with one or more components of system 100A. In some examples, the clinician, (e.g., physician, technician, surgeon, electrophysiologist, or other clinician) may interact with clinician programmer 104A to communicate with IMD 106A. For example, the clinician may retrieve physiological or diagnostic information from IMD 106A via clinician programmer 104A. As another example, the clinician may interact with clinician programmer 104A to program IMD 106A, e.g., select values that define electrical stimulation generated and delivered by IMD 106A, select other operational parameters of IMD 106A, or the like. For example, as described herein, a clinician may select variations of parameters, which pulse variation patterns to use, how to define the sequence of different pulse trains, adjust a sequence of pulse trains, remove, add, or replace one or more pulse trains, or any other type of stimulation programming. As another example, the clinician may use clinician programmer 104A to retrieve information from IMD 106A regarding the performance or integrity of IMD 106A or other components of system 10, such as lead 114 or a power source of IMD 106A. In some examples, this information may be presented to the clinician as an alert if a system condition that may affect the efficacy of therapy is detected.

In some examples, a clinician may use clinician programmer 104A to create stimulation programs for electrical stimulation (generated and delivered by IMD 106A) as therapy to treat bladder dysfunction of patient 112. In some examples, the clinician programmer 104A transmits the stimulation programs to IMD 106A for storage in a memory of IMD 106A. Clinician programmer 104A may be configured to have additional functionality and/or control of IMD 106A than the patient programmer 104B.

Patient programmer 104B facilitates interaction of patient 112 with one or more components of system 100A. In some examples, patient 112 may interact with patient programmer 104B to control IMD 106A to deliver electrical stimulation, to manually abort the delivery of electrical stimulation by IMD 106A, or to inhibit the delivery of electrical stimulation by IMD 106A. Patient 112 may, for example, use a keypad or touch screen of patient programmer 104B to cause IMD 106A to deliver electrical stimulation, e.g., to activate one or more stimulation programs, or the like.

IMD 106A and device 104 may communicate via wireless communication using any techniques known in the art. Examples of communication techniques may include, for example, low frequency or radiofrequency (RF) telemetry, or Bluetooth. Other techniques are also contemplated. In some examples, device 104 may include a programming head that may be placed proximate to the patient's body near the IMD 106A implant site in order to improve the quality or security of communication between IMD 106A and device 104.

Device 104 and IMD 106A may each output a set of advertisements to establish a communication session between device 104 and IMD 106A. In some examples, device 104 and IMD 106A may connect based on a data transmission interval, such as an advertising interval. As used herein, an advertisement for a connection may include one or more packets (e.g., one or more advertisement packets) that include information for connecting devices. The advertisement may be sent to inquire whether communication should be initiated. An advertising device (e.g., IMD 106A) may communicate (e.g., output, send, etc.) the advertisement on an advertising channels. An advertising interval may refer to a rate at which an advertising device outputs the advertisement. For example, device 104 may periodically output an advertisement for connection (e.g., an advertisement packet) at an advertising interval (e.g., less than 100 ms, 100 ms to 500 ms, 1 second, more than 1 second, etc.) with a random delay. The advertisement for connection may include information on how to connect with the advertising device, such as, for example, one or more of: (1) media access control (MAC) addresses for the medical device and external device; (2) a real time-point in time for the transfer to start; (3) an indication of a starting frequency; (4) an indication of a hop set; (5) a connection interval; or (6) a connection latency.

In accordance with the techniques of the disclosure, IMD 106A may include telemetry circuitry 130 that is configured to be inactive (e.g., “sleeping,” idle, etc.). When inactive, telemetry circuitry 130 may have limited functionality and in turn draw little to no current, thereby reducing energy consumption. For example, when telemetry circuitry 130 is inactive, telemetry circuitry 130 may only monitor for an activation signal, such as an in-band sting (described in greater detail below). Processing circuitry of IMD 106A may be inactive as well to reduce energy consumption. When telemetry circuitry 130 detects an activation signal, telemetry circuitry 130 may activate and “wake up” the processing circuitry to allow for, e.g., a programming session. Based on the activation signal from telemetry circuitry 130, the processing circuitry may wake up in operation mode, development mode, test mode, etc. Telemetry circuitry 130 may go to sleep after a certain period of inactive communication.

When telemetry circuitry 130 is inactive, telemetry circuitry 130 may not decode signals, such as RF signals (e.g., a wireless electromagnetic signal used as a form of communication in the frequency range from around 20 kHz to around 300 GHz), until wake circuitry 138 of IMD 106A detects the activation signal. Telemetry circuitry 130 may be configured to activate (e.g., “wake up”) or transition to an active state in response to receiving an activation signal from wake circuitry 138 of IMD 106A. Wake circuitry 138 may output an activation signal to telemetry circuitry 130 in response to receiving a first set of advertisements from device 104 (e.g., a patient programmer, a clinician programmer, etc.).

When active, telemetry circuitry 130 may be configured to establish a communication session with device 104 by outputting a second set of advertisements. The second set of advertisements may be at the same frequency band as the first set of advertisements such that the mechanism for activating telemetry circuitry 130 is in-band. An in-band sting mechanism may advantageously allow for reduced design complexity and device cost. For example, an in-band sting mechanism as described herein may allow for removal of coils and decrease the variety of components (e.g., the number of different types of antennas) for IMD 106A and/or device 104. For example, logic components for decoding inductive telemetry protocol may be removed from IMD 106A. Further, the reduced design complexity may allow for increased usage of generic (as opposed to custom) components, such as off-the-shelf phones, modules, and other components.

As used herein, a sting (e.g., an in-band sting or an out-of-band sting) may refer to a burst or a patterned burst for communication. In general, stinging may not necessarily include advertisements or handshaking. For instance, an inductive telemetry device may be placed near IMD 106A to cause IMD 106A to establish a connection more quickly than if not for the inductive sting. In this instance, IMD 106A may “ping” other devices, for example, by outputting an advertisement that another device (e.g., an external device) may listen for in order to determine if there is a device out there for exchanging communications. The ping may be a telemetry output from IMD 106A and may be in-band (e.g., a Bluetooth™ low energy (BLE) advertisement).

IMD 106A may include wake circuitry 138. Wake circuitry 138 may be electrically coupled to an antenna of IMD 106A such that wake circuitry 138 may receive signals from the antenna. For example, the antenna may receive a signal associated with a first set of advertisements from device 104, and the antenna may transmit the signal to wake circuitry 138. Device 104 may have transmitted the first set of advertisements at a frequency band, such as ISM frequency bands from 2.402 GHz to 2.48 GHz. The antenna may be configured to operate at the frequency band of the first set of advertisements (e.g., from 2.402 GHz to 2.48 GHz). That is, one or more parameters of the antenna may be configured for the frequency band. For example, the length of the antenna may correspond to a resonant length for the frequency band (e.g., 20 millimeters (mm) to 25 mm for a frequency band centered around 2.45 GHz). Wake circuitry 138 may receive the first set of advertisements at the frequency band. Other frequency bands, such as frequency bands in the kilohertz (KHz) or megahertz (MHz) range, are contemplated by this disclosure.

Responsive to receiving a first set of advertisements from device 104, wake circuitry 138 may output a set of pulses (e.g., voltage signals) based on the first set of advertisements. Wake circuitry 138 may use the first set of advertisements to generate the set of pulses. For example, the set of pulses may be based on parameters of the first set of advertisements, such as the advertising interval, signal strength (e.g., voltage), etc.

Wake circuitry 138 may be configured to harvest energy from a signal, such as the signal carrying the first set of advertisements. For instance, wake circuitry 138 may use energy from an RF signal that carries the first set of advertisements to power operations of wake circuitry 138. As a result, wake circuitry 138 may use less (or even no) current from energy source of IMD 106A, increasing energy efficiency. In some examples, wake circuitry 138 may include an energy harvesting module, such as a passive RF energy harvester configured to harvest energy from an RF signal received by IMD 106A.

Wake circuitry 138 may be configured to sting telemetry circuitry 130 in response to a data pattern, e.g., an advertising pattern, of the set of pulses satisfying a data pattern requirement, e.g., an advertising pattern requirement. Wake circuitry 138 may detect the advertising pattern using the set of pulses. For instance, wake circuitry 138 may be configured to detect, using the set of pulses, advertising intervals (e.g., less than 100 ms, 100 ms to 500 ms, 1 second, more than 1 second, etc.) of the advertising pattern. As an example, wake circuitry 138 may detect, and the advertising pattern may include, a first advertising interval of 100 ms, a second advertising interval of 150 ms, etc.

In another example, wake circuitry 138 may be configured to compare a voltage of each pulse of the set of pulses to a voltage threshold and detect an advertising pattern based on the comparison of the voltage of each pulse of the set of pulses to a voltage threshold. For instance, wake circuitry 138 may output a ‘high’ signal when the voltage of a pulse is greater than the voltage threshold and output a ‘low’ signal when the voltage of a pulse is less than or equal to the voltage threshold. Wake circuitry 138 may process the signal to detect an advertising pattern (e.g., a pattern in the intervals between the high signals).

Wake circuitry 138 may determine whether the advertising pattern matches an advertising pattern requirement. For example, an advertising pattern detection module may determine whether the intervals between high signals match a predetermined advertising pattern. Responsive to the advertising pattern matching a predetermined advertising pattern, wake circuitry 138 may determine that the advertising pattern of the set of pulses (which are based on the first set of advertisements from device 104) satisfies the advertising pattern requirement. On the other hand, responsive to the advertising pattern not matching a predetermined advertising pattern, wake circuitry 138 may determine that the advertising pattern of the set of pulses does not satisfy the advertising pattern requirement.

Responsive to determining that the advertising pattern satisfies an advertising pattern requirement, wake circuitry 138 may output an activation signal that causes telemetry circuitry 130 to transition from an inactive state to an active state. In other words, wake circuitry 138 may sting telemetry circuitry 130 to “wake” telemetry circuitry 130. Stinging telemetry circuitry 130 in response to detection of a pattern may help avoid waking telemetry circuitry 130 whenever a signal that is within a particular frequency band is detected. For example, many devices other than device 104 may output a signal that is at the 2.402 GHz to 2.48 GHz frequency band. However, only device 104 associated with IMD 106A may transmit or generate a set of advertisements having a pattern that satisfying the advertising pattern requirement. As a result, only the set of advertisements from device 104 may lead to activation of telemetry circuitry 130, helping reduce or avoid unnecessary or unwarranted activation.

When active, telemetry circuitry 130 may be configured to establish a communication session with device 104. Telemetry circuitry 130 may establish a communication session by using a second set of advertisements (e.g., by transmitting the second set of advertisements to device 104 via the antenna). Telemetry circuitry 130 may transmit the second set of advertisements at the same frequency bands as the first set of advertisements. For example, if device 104 transmitted the first set of advertisements at a 2.402 GHz to 2.48 GHz frequency band (e.g., per the BLE protocol), then telemetry circuitry 130 may establish a communication session by transmitting a second set of advertisements at the 2.402 GHz to 2.48 GHz frequency band. In some examples, telemetry circuitry 130 may receive electrical stimulation information from device 104 using the communication session. IMD 106A may be configured to provide therapy based on the electrical stimulation information.

The first set of advertisements may be compliant with a communication protocol, such as BLE. The second set of advertisements may be compliant with the communication protocol for the first set of advertisements. The first set of advertisements and the second set of advertisements may be both advertised on a set of frequency channels assigned for advertisements by the communication protocol with which the first set of advertisements and the second set of advertisements are compliant. In some examples, the second set of advertisements may include a hop set of frequencies. In such examples, telemetry circuitry 130 may be configured to establish the communication session according to the hop set of frequencies.

The techniques of this disclosure may enable system 100A to activate telemetry circuitry 130 only when device 104 is within range and attempting to connect to IMD 106A, in turn reducing the time when telemetry circuitry 130 is active and consuming higher rates of current.

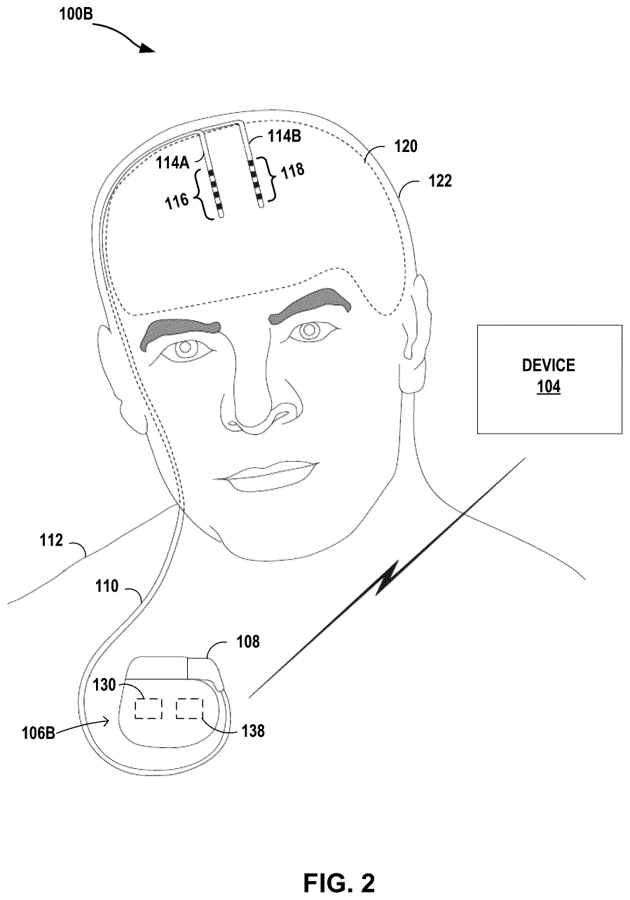

FIG. 2 is a conceptual diagram illustrating an example system 100B that includes an IMD 106B configured to deliver adaptive DBS to a patient 112. System 100B may employ energy conservation techniques in accordance with this disclosure, as described above with respect to FIG. 1. That is, system 100B may be substantially similar to system 100A (collectively, “system 100”) except for any differences described herein. Although the examples described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable DBS system for purposes of illustration, but without limitation as to other types of medical devices or other therapeutic applications of stimulation. In some examples, one or more components of system 100B may be configured to deliver one or more of deep brain stimulation (DBS), spinal cord stimulation (SCS), sacral neuromodulation (SNS), targeted drug delivery (TDD), pelvic stimulation, gastric stimulation, or peripheral nerve field stimulation (PNFS), tibial nerve stimulation (TNS), or any other stimulation therapy capable of treating a condition of patient 112.

DBS may be adaptive in the sense that IMD 106B may adjust, increase, or decrease the magnitude of one or more parameters of the DBS in response to changes in patient activity or movement, a severity of one or more symptoms of a disease of the patient, a presence of one or more side effects due to the DBS, or one or more sensed signals of the patient. For instance, one example of system 100B is a bi-directional DBS system with capabilities to both deliver stimulation and sense intrinsic neuronal signals. System 100B may provide for “closed-loop” therapy where IMD 106B may continuously monitor the state of certain biomarker signals and deliver stimulation according to pre-programmed routines based on the biomarker signals.

System 100B may be configured to treat a patient condition, such as a movement disorder, neurodegenerative impairment, a mood disorder, or a seizure disorder of patient 112. Patient 112 ordinarily is a human patient. In some cases, however, system 100B may be applied to other mammalian or non-mammalian, non-human patients. While movement disorders and neurodegenerative impairment are primarily referred to herein, in other examples, therapy system 100B may provide therapy to manage symptoms of other patient conditions, such as, but not limited to, seizure disorders (e.g., epilepsy) or mood (or psychological) disorders (e.g., major depressive disorder (MDD), bipolar disorder, anxiety disorders, post-traumatic stress disorder, dysthymic disorder, and obsessive-compulsive disorder (OCD)) as well as, for example, neural control of prosthetic devices or stimulation to provide sensory feedback to the patients. At least some of these disorders may be manifested in one or more patient movement behaviors. A movement disorder or other neurodegenerative impairment may include symptoms such as, for example, muscle control impairment, motion impairment or other movement problems, such as rigidity, spasticity, bradykinesia, rhythmic hyperkinesia, nonrhythmic hyperkinesia, and akinesia. In some cases, the movement disorder may be a symptom of Parkinson's disease. However, the movement disorder may be attributable to other patient conditions.

System 100B may include device 104 associated with IMD 106B, lead extension 110, one or more leads 114A and 114B with respective sets of one or more electrodes 116, 118. In the example shown in FIG. 1, electrodes 116, 118 of leads 114A, 114B are positioned to deliver electrical stimulation to a tissue site within brain 120, such as a deep brain site under the dura mater of brain 120 of patient 112. In some examples, delivery of stimulation to one or more regions of brain 120, such as the subthalamic nucleus, globus pallidus or thalamus, may be an effective treatment to manage movement disorders, such as Parkinson's disease. Some or all of electrodes 116, 118 also may be positioned to sense neurological brain signals within brain 120 of patient 112. In some examples, some of electrodes 116, 118 may be configured to sense neurological brain signals and others of electrodes 116, 118 may be configured to deliver adaptive electrical stimulation to brain 120. In other examples, all of electrodes 116, 118 are configured to both sense neurological brain signals and deliver adaptive electrical stimulation to brain 120. In some examples, unipolar stimulation may be possible where one electrode is on the housing of IMD 106B.

IMD 106B includes a therapy module (e.g., which may include processing circuitry, signal generation circuitry or other electrical circuitry configured to perform the functions attributed to IMD 106B) that includes a stimulation generator configured to generate and deliver electrical stimulation therapy to patient 112 via a subset of electrodes 116, 118 of leads 114A and 114B, respectively. The subset of electrodes 116, 118 that are used to deliver electrical stimulation to patient 112, and, in some cases, the polarity of the subset of electrodes 116, 118, may be referred to as a stimulation electrode combination. As described in further detail below, the stimulation electrode combination can be selected for a particular patient 112 and target tissue site (e.g., selected based on the patient condition). The group of electrodes 116, 118 includes at least one electrode and can include a plurality of electrodes. In some examples, the plurality of electrodes 116 and/or 118 may have a complex electrode geometry such that two or more electrodes are located at different positions around the perimeter of the respective lead.

In some examples, neurological signals sensed within brain 120 may reflect changes in electrical current produced by the sum of electrical potential differences across brain tissue. Examples of neurological brain signals include, but are not limited to, bioelectric signals generated from local field potentials (LFP) sensed within one or more regions of brain 120. Electroencephalogram (EEG) signal or an electrocorticogram (ECoG) signal are also examples of bioelectric signals. For example, neurons generate the bioelectric signals, and if measured at depth, the bioelectric signals are LFP; if measured on the cerebral cortex, the bioelectric signals are ECoG; if measured on the scalp, the bioelectric signals are EEG. In this disclosure, the term “oscillatory signal source” may be used to describe one example of a signal source that generates bioelectric signals. However, the bioelectric signals are not limited to oscillatory signals. For example purposes, the techniques are described with oscillatory bioelectric signals from an oscillatory signal source.

In some examples, the neurological brain signals that are used to select a stimulation electrode combination may be sensed within the same region of brain 120 as the target tissue site for the electrical stimulation. As previously indicated, these tissue sites may include tissue sites within anatomical structures such as the thalamus, subthalamic nucleus or globus pallidus of brain 120, as well as other target tissue sites. The specific target tissue sites and/or regions within brain 120 may be selected based on the patient condition. Thus, in some examples, both stimulation electrode combinations and sense electrode combinations may be selected from the same set of electrodes 116, 118. In other examples, the electrodes used for delivering electrical stimulation may be different than or the same as the electrodes used for sensing neurological brain signals.

IMD 106B may be implanted within a subcutaneous pocket above the clavicle, or, alternatively, on or within cranium 122 or at any other suitable site within patient 112. Generally, IMD 106B is constructed of a biocompatible material that resists corrosion and degradation from bodily fluids. IMD 106B may comprise a hermetic housing to substantially enclose components, such as a processor, therapy module, and memory.

As shown in FIG. 2, implanted lead extension 110 is coupled to IMD 106B via connector 108 (also referred to as a connector block or a header of IMD 106B). In the example of FIG. 2, lead extension 110 traverses from the implant site of IMD 106B and along the neck of patient 112 to cranium 122 of patient 112 to access brain 120. In the example shown in FIG. 2, leads 114A and 114B (collectively “leads 114”) are implanted within the right and left hemispheres (or in just one hemisphere in some examples), respectively, of patient 112 in order to deliver electrical stimulation to one or more regions of brain 120, which may be selected based on the patient condition or disorder controlled by therapy system 100B. The specific target tissue site and the stimulation electrodes used to deliver stimulation to the target tissue site, however, may be selected, e.g., according to the identified patient behaviors and/or other sensed patient parameters.

IMD 106B includes a memory to store a plurality of therapy programs that each define a set of therapy parameter values. In some examples, IMD 106B may select a therapy program from the memory based on various parameters, such as sensed patient parameters and the identified patient behaviors. IMD 106B may generate electrical stimulation based on the parameters of the selected therapy program to manage the patient symptoms associated with a movement disorder.

Therapy system 100B may be implemented to provide chronic stimulation therapy to patient 112 over the course of several months or years. However, system 100B may also be employed on a trial basis to evaluate therapy before committing to full implantation. If implemented temporarily, some components of system 100B may not be implanted within patient 112. For example, patient 112 may be fitted with an external medical device, such as a trial stimulator, rather than IMD 106B. The external medical device may be coupled to percutaneous leads or to implanted leads via a percutaneous extension. If the trial stimulator indicates DBS system 100B provides effective treatment to patient 112, the clinician may implant a chronic stimulator within patient 112 for relatively long-term treatment.

Although IMD 106B and system 100B are described as delivering electrical stimulation therapy to brain 120, IMD 106B and system 100B may be configured to direct electrical stimulation to other anatomical regions of patient 112. Further, an IMD may provide other electrical stimulation such as spinal cord stimulation to treat a movement disorder. For example, in some examples, an IMD may be configured to deliver one or more of deep brain stimulation (DBS), spinal cord stimulation (SCS), sacral neuromodulation (SNS), targeted drug delivery (TDD), pelvic stimulation, gastric stimulation, peripheral nerve field stimulation (PNFS), tibial nerve stimulation (TNS), or any other stimulation therapy capable of treating a condition of patient 112.

IMD 106B may include telemetry circuitry 130 that is configured to be inactive (e.g., “sleeping,” idle, etc.). When telemetry circuitry 130 is inactive, telemetry circuitry 130 may not decode signals, such as RF signals, until wake circuitry 138 of IMD 106B detects an in-band sting. Telemetry circuitry 130 may be configured to activate or transition to an active state in response to receiving an activation signal from wake circuitry 138 of IMD 106B. Wake circuitry 138 may output an activation signal to telemetry circuitry 130 in response to receiving a first set of advertisements from device 104. When active, telemetry circuitry 130 may be configured to establish a communication session with device 104 by outputting a second set of advertisements. The second set of advertisements may be at the same frequency band as the first set of advertisements such that the mechanism for activating telemetry circuitry 130 is in-band.

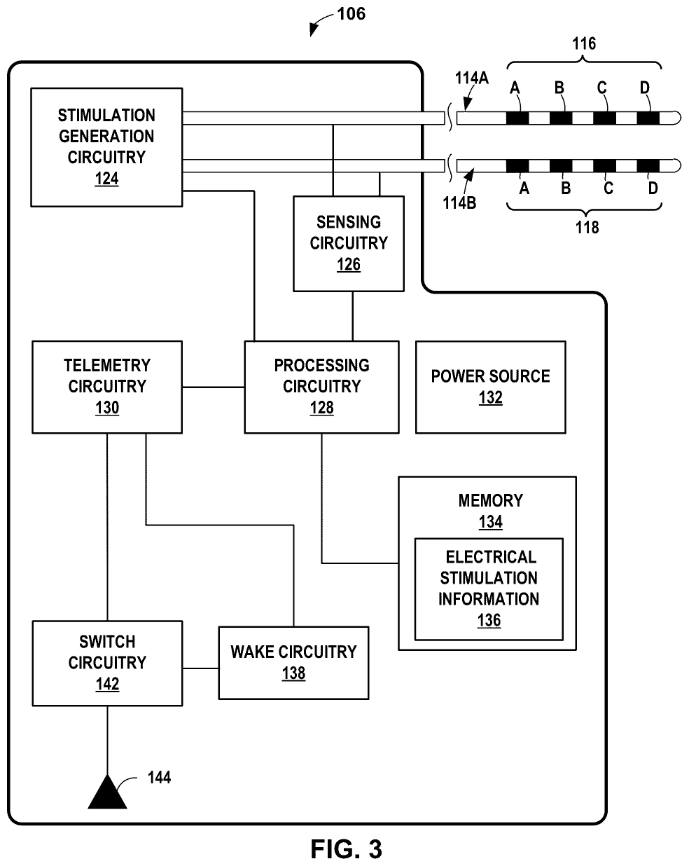

FIG. 3 is a block diagram of example IMD 106B of FIG. 2 for delivering stimulation therapy. In the example shown in FIG. 3, IMD 106B includes processing circuitry 128, memory 134, wake circuitry 138, wake circuitry 138, switch circuitry 142, stimulation generation circuitry 124, sensing circuitry 126, and telemetry circuitry 130, a power source 132, and an antenna 144. Each of these circuits may be or include electrical circuitry configured to perform the functions attributed to each respective circuit.

Memory 134 may include any volatile or non-volatile media, such as a random-access memory (RAM), read only memory (ROM), non-volatile RAM (NVRAM), ferroelectric RAM (FRAM), electrically erasable programmable ROM (EEPROM), flash memory, and the like. Memory 134 may store computer-readable instructions that, when executed by processing circuitry 128, cause IMD 106B to perform various functions. Memory 134 may be a storage device or other non-transitory medium.

In the example shown in FIG. 2, memory 134 stores electrical stimulation information 136. Electrical stimulation information 136 may include electrical stimulation information (e.g., a therapy parameter set), such as a stimulation electrode combination, electrode polarity, current or voltage amplitude, pulse width, and pulse rate. In some examples, individual therapy programs may be stored as a therapy group, which defines a set of therapy programs with which stimulation may be generated. The stimulation signals defined by the therapy programs of the therapy group may be delivered together on an overlapping or non-overlapping (e.g., time-interleaved) basis.

Stimulation generation circuitry 124, under the control of processing circuitry 128, generates stimulation signals for delivery to patient 112 via selected combinations of one or more electrodes 116, 118. An example range of electrical stimulation parameters believed to be effective in DBS to manage a movement disorder of patient include:
1. Pulse Rate, i.e., Frequency: between approximately 40 Hertz and approximately 500 Hertz, such as between approximately 90 to 170 Hertz or such as approximately 90 Hertz.
2. In the case of a voltage controlled system, Voltage Amplitude: between approximately 0.1 volts and approximately 5 volts, such as between approximately 2 volts and approximately 3 volts.
3. In the case of a current controlled system, Current Amplitude: between approximately 1 milliamps to approximately 8 milliamps, such as between approximately 1.0 milliamps and approximately 3 milliamps.
4. Pulse Width: between approximately 20 microseconds and approximately 500 microseconds, such as between approximately 50 microseconds and approximately 200 microseconds.

Accordingly, in some examples, stimulation generation circuitry 124 may generate electrical stimulation signals in accordance with the electrical stimulation parameters noted above, which may be examples of electrical stimulation information. Other ranges of therapy parameter values may also be useful, and may depend on the target stimulation site within patient 112. While stimulation pulses are described, stimulation signals may be of any form, such as continuous-time signals (e.g., sine waves) or the like.

Processing circuitry 128 may include fixed function processing circuitry and/or programmable processing circuitry, and may comprise, for example, any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 128 herein may be embodied as firmware, hardware, software or any combination thereof. Processing circuitry 128 may control stimulation generation circuitry 124 according to electrical stimulation information 136 stored in memory 134 to apply particular stimulation parameter values specified by one or more of programs, such as voltage amplitude or current amplitude, pulse width, and/or pulse rate. In some examples, processing circuitry 128 may include one or more of wake circuitry 138 or wake circuitry 138. In other examples, wake circuitry 138 and wake circuitry 138 may be separate from processing circuitry 128.

In the example shown in FIG. 3, the set of electrodes 116 includes electrodes 116A, 116B, 116C, and 116D, and the set of electrodes 118 includes electrodes 118A, 118B, 118C, and 118D. Processing circuitry 128 may control individual voltage or current sources and sinks coupled to respective electrodes 116, 118, functioning as cathodes or anodes, to delivery stimulation signals to patient tissue.

Stimulation generation circuitry 124 may be a single channel or multi-channel stimulation generator. In particular, stimulation generation circuitry 124 may be capable of delivering a single stimulation pulse, multiple stimulation pulses, or a continuous signal at a given time via a single electrode combination or multiple stimulation pulses at a given time via multiple electrode combinations. For example, as mentioned above, stimulation generation circuitry 124 may comprise multiple voltage or current sources and sinks that are coupled to respective electrodes to drive the electrodes as cathodes or anodes simultaneously or at different times. In some examples, stimulation generation circuitry 124 may be configured to deliver multiple channels on a time-interleaved basis.

Electrodes 116, 118 on respective one or more leads 114 may be constructed of a variety of different designs. For example, one or both of leads 114 may include two or more electrodes at each longitudinal location along the length of the lead, such as multiple electrodes, e.g., arranged as segments, at different perimeter locations around the perimeter of the lead at each of the locations A, B, C, and D.

As an example, one or both of leads 114 may include radially-segmented DBS arrays (rDBSA) of electrodes. In the rDBSA, as one example, there may be a first ring electrode of electrodes 116 around the perimeter of lead 114A at a first longitudinal location on lead 114A (e.g., location A). Below the first ring electrode, there may be three segmented electrodes of electrodes 116 around the perimeter of lead 114A at a second longitudinal location on lead 114A (e.g., location B). Below the three segmented electrodes, there may be another set of three segmented electrodes of electrodes 116 around the perimeter of lead 114A at a third longitudinal location of lead 114A (e.g., location C). Below the three segmented electrodes, there may be a second ring electrode of electrodes 116 around the perimeter of lead 114A (e.g., location D). Electrodes 118 may be similarly positioned along lead 114B.

The above is one example of the rDBSA array of electrodes, and the example techniques should not be considered limited to such an example. There may be other configurations of electrodes for DBS. Moreover, the example techniques are not limited to DBS, and other electrode configurations are possible.

In one example, the electrodes 116, 118 may be electrically coupled to a connector at the proximal end of the lead. In another example, each of the electrodes 116, 118 of the leads 114 may be electrodes deposited on a thin film. The thin film may include an electrically conductive trace for each electrode that runs the length of the thin film to a proximal end connector. The thin film may then be wrapped (e.g., a helical wrap) around an internal member to form the leads 114. These and other constructions may be used to create a lead with a complex electrode geometry.

Although sensing circuitry 126 is incorporated into a common housing with stimulation generation circuitry 124 and processing circuitry 128 in FIG. 3, in other examples, sensing circuitry 126 may be in a separate housing from IMD 106B and may communicate with processing circuitry 128 via wired or wireless communication techniques. Sensing circuitry 126 may sense one or more bioelectric signals of a brain or nerve of a patient and stimulation generation circuitry may generate the electrical stimulation based on the one or more bioelectric signals. However, techniques described herein may apply to all types of medical devices, particularly those that have a patient programmer where, for purposes of user experience, the response of the interaction between the patient and the patient programmer should appear to be minimal (e.g., no delay, a 1-5 second delay, 5-10 second delay, less than a 30 second delay, etc.) when the patient requests to perform an action on the patient programmer. This may directly apply to neuromodulation devices such as, for example, spinal cord stimulation (SCS) devices, deep brain stimulation (DBS) devices, sacral nerve stimulation (SNS) devices, or peripheral nerve stimulation therapy.

Example neurological brain signals include, but are not limited to, a signal generated from local field potentials (LFPs) within one or more regions of brain 120. EEG and ECoG signals are examples of local field potentials that may be measured within brain 120. LFPs, EEG and EcoG may be different measurements of the same bioelectric signals in the brain. The neurons generate the signals, and if measured at depth, it is LFP, if measured on the coretex, it is EcoG, if on the scalp, it is EEG. In general, the bioelectric signals may be formed by one or more oscillatory signal sources. The set of electrodes 116 and 118 that are most proximate to the oscillatory signal sources are good candidates to use for delivering therapy. Additionally, sensing circuitry 126 may sense EMG, ECAP, Electrogram Guided Myocardial Advanced Phenotypings (EMAPs), or other neural or physiological signals generated in response to a stimulation or in response to other evocation, muscle movement, user-evoked signals, etc. For example, incontinence therapy may involve sensing muscle and/or neural signals.

Telemetry circuitry 130 may be configured to support wireless communication. For example, telemetry circuitry 130 may be configured to support wireless communication using Bluetooth™ (e.g., BLE and other versions of Bluetooth™, including future versions of Bluetooth™), Wi-Fi™, Near-Field Communication (NFC), Near Field Magnetic Induction (NFMI), Long Term Evolution, 5^{th} generation (LTE/5G), or MedRadio (MICS: Medical Implant Communication Service, MEDS: Medical External Device Service, MBAD: Medical Body Area Network)) between IMD 106B and an device 104 or another computing device under the control of processing circuitry 128. In some examples, telemetry circuitry 130 supports a telemetry frequency that may correspond to a high frequency or radio frequency, which may be a radio frequency established via Bluetooth, Wi-Fi, Near-Field Communication (NFC), 175 KHz inductive telemetry, or MICS, for example. Telemetry circuitry 130 may be configured to receive an inductive sting. Processing circuitry 128 of IMD 106B may receive, as updates to programs (e.g., at least one program parameter), values for various stimulation parameters such as magnitude and electrode combination, from device 104 via telemetry circuitry 130. The updates to the therapy programs may be stored within electrical stimulation information 136 portion of memory 134. Telemetry circuitry 130 in IMD 106B, as well as telemetry modules in other devices and systems described herein, such as device 104, may accomplish communication by radiofrequency (RF) communication techniques (e.g., Bluetooth, Wi-Fi, Near-Field Communication (NFC), or MICS). In addition, telemetry circuitry 130 may communicate with external medical device 104 via proximal inductive interaction of IMD 106B with device 104. Accordingly, telemetry circuitry 130 may send information to device 104 on a continuous basis, at periodic intervals, or upon request from IMD 106B or device 104.

Telemetry circuitry 130 may periodically output an advertisement for a connection at an advertising interval (e.g., less than 100 ms, 100 ms to 500 ms, 1 second, more than 1 second, etc.) with an optional random delay. The advertisement may include information on how to connect with the advertising device, such as, for example, one or more of: (1) media access control (MAC) addresses for the medical device and external device; (2) a real time-point in time for the transfer to start; (3) an indication of a starting frequency; (4) an indication of a hop set; (5) a connection interval; or (6) a connection latency. In some examples, telemetry circuitry 130 may receive the advertisement and connect with another device (e.g., device 104) using the received advertisement (e.g., using a starting frequency and hop set of the received advertising packet). Telemetry circuitry 130 may be electrically connected to switch circuitry 142 and wake circuitry 138.

Antenna 144 may be configured to operate at the frequency band of the first set of advertisements and the second set of advertisements (e.g., from 2.402 GHz to 2.48 GHz). That is, one or more parameters of the antenna may be configured for the frequency band. For example, the length of the antenna may correspond to a resonant length for the frequency band (e.g., 20 millimeters (mm) to 25 mm for a frequency band centered around 2.45 GHz). Although shown in FIG. 3 as completely within a housing of IMD 106B, antenna 144 may be partially or entirely outside of the housing of IMD 106B.

In some examples, IMD 106B may include switch circuitry 142. Switch circuitry 142 may be configured to electrically connect and disconnect telemetry circuitry 130 and antenna 144. Switch circuitry 142 may be configured to electrically connect and disconnect wake circuitry 138 and antenna 144. Switch circuitry 142 may also be configured to electrically connect and disconnect telemetry circuitry 130 and antenna 144. For example, processing circuitry 128 of IMD 106B may be configured to cause switch circuitry 142 to cause switch circuitry 142 to electrically connect wake circuitry 138 and antenna 144 and electrically disconnect telemetry circuitry 130 and antenna 144 (e.g., by default, in response to a termination of the communication session, etc.). This may prevent attenuation of the signal strength of a first set of advertisements received by antenna 144 that may otherwise occur due to the signal carrying the first set of advertisements splitting between telemetry circuitry 130 and wake circuitry 138. For example, when telemetry circuitry 130 and antenna 144 are electrically disconnected, the signal may only flow from antenna 144 to wake circuitry 138 such that wake circuitry 138 receives the signal at full strength. This may increase the range IMD 106B may detect a signal from device 104.

Additionally, processing circuitry 128 of IMD 106B may be configured to cause switch circuitry 142 to electrically connect telemetry circuitry 130 and antenna 144 and electrically disconnect wake circuitry 138 and antenna 144 in response to a determination that the advertising pattern of the first set of frequencies satisfies an advertising pattern requirement. This may prevent attenuation of the signal strength of the outputted second set of advertisements for similar reasons described above. In this way, telemetry circuitry 130 may use antenna 144 to output the second set of advertisements at full strength to establish a communication session.

Examples of switching circuitry 142 may include, but are not limited to, a silicon-controlled rectifier (SCR), a Field Effect Transistor (FET), and a bipolar junction transistor (BJT). Examples of FETs may include, but are not limited to, a junction field-effect transistor (JFET), a metal-oxide-semiconductor FET (MOSFET), a dual-gate MOSFET, an insulated-gate bipolar transistor (IGBT), any other type of FET, or any combination of the same. Examples of MOSFETS may include, but are not limited to, a depletion mode p-channel MOSFET (PMOS), an enhancement mode PMOS, depletion mode n-channel MOSFET (NMOS), an enhancement mode NMOS, a double-diffused MOSFET (DMOS), any other type of MOSFET, or any combination of the same. Examples of BJTs may include, but are not limited to, PNP, NPN, heterojunction, or any other type of BJT, or any combination of the same. It should be understood that switching elements may be high-side or low-side switching elements. Additionally, switching elements may be voltage-controlled and/or current-controlled.

Power source 132 delivers operating power to various components of IMD 106B. Power source 132 may include a small rechargeable or non-rechargeable battery and a power generation circuit to produce the operating power. Recharging may be accomplished through proximal inductive interaction between an external charger and an inductive charging coil within IMD 106B. In some examples, power requirements may be small enough to allow IMD 106B to utilize patient motion and implement a kinetic energy-scavenging device to trickle charge a rechargeable battery. In other examples, traditional batteries may be used for a limited period of time.

Processing circuitry 128 of IMD 106B may sense, via electrodes 116, 118 interposed along leads 114 (and sensing circuitry 126), one or more bioelectric signals of brain 120 of patient 112. Further, processing circuitry 128 of IMD 106B may deliver, via electrodes 116, 118 (and stimulation generation circuitry 124), electrical stimulation therapy to patient 112 based on the sensed one or more bioelectric signals of brain 120. The adaptive DBS therapy is defined by electrical stimulation information 136. For example, electrical stimulation information 136 may include a current amplitude (for a current-controlled system) or a voltage amplitude (for a voltage-controlled system), a pulse rate or frequency, and a pulse width, or a number of pulses per cycle. In examples where the electrical stimulation is delivered according to a “burst” of pulses, or a series of electrical pulses defined by an “on-time” and an “off-time,” the one or more parameters may further define one or more of a number of pulses per burst, an on-time, and an off-time. Processing circuitry 128, via electrodes 116, 118, delivers to patient 112 adaptive DBS and may adjust one or more parameters defining the electrical stimulation based on corresponding parameters of the sensed one or more bioelectric signals of brain 120.

In some examples, processing circuitry 128 may continuously measure the one or more bioelectric signals in real time. In other examples, processing circuitry 128 may periodically sample the one or more bioelectric signals according to a predetermined frequency or after a predetermined amount of time. In some examples, processing circuitry 128 may periodically sample the signal at a frequency of approximately 150 Hertz.

Telemetry circuitry 130 may be configured to output an advertisement, such as, advertisement for a wireless communication session or advertisement compliant with another protocol. For example, telemetry circuitry 130 may output the advertisement at an advertising interval. The advertising interval may include a BLE advertising interval. Telemetry circuitry 130 may ping device 104. For example, telemetry circuitry 130 may output an advertisement or advertisement package that device 104 or another device listens for in order to determine if there is a device for exchanging communications. In some examples, telemetry circuitry 130 may output the advertisement in response to a sting and/or may periodically output the advertisement using an advertising interval (e.g., with a random delay).

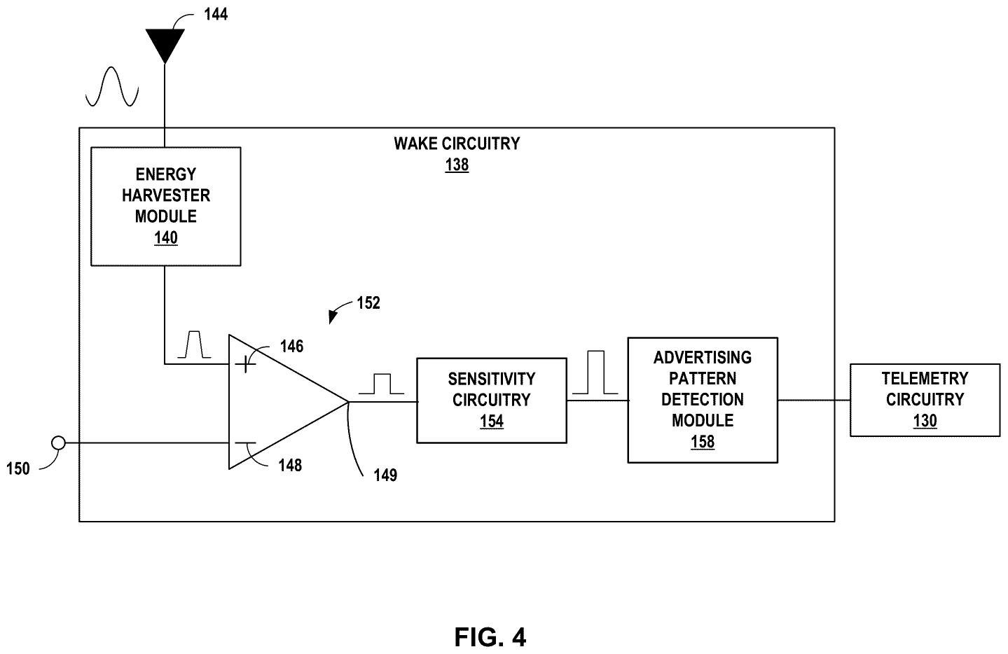

FIG. 4 is a block diagram of a portion of IMD 106A and/or IMD 106B (collectively, IMD 106). As shown in FIG. 3, IMD 106 include antenna 144, wake circuitry 138, an energy harvester module 140, and telemetry circuitry 130. As shown in FIG. 4, wake circuitry 138 includes a comparator 152, sensitivity circuitry 154, and an advertising pattern detection module 158.

Antenna 144 may receive a first set of advertisements from device 104. Antenna 144 may send the first set of advertisements to energy harvester module 140. Energy harvester module 140 (e.g., a passive RF energy harvester) may be configured to harvest energy from a signal received by IMD 106A. For example, energy harvester module 140 may store or use the power generated by a carrier wave indicating the signal. In any case, responsive to receiving the first set of advertisements, energy harvester module 140 may output a set of pulses based on the first set of advertisements. In some examples, energy harvester module 140 may only output a pulse if the RF signal carrying the first set of advertisements is within a frequency band corresponding to a specific protocol (i.e., in-band). The voltage of the pulses may correspond to a strength of the signal (e.g., a distance and/or power output of the transmitter). In this way, energy harvester module 140 may use the first set of advertisements to generate a set of pulses. The set of pulses may be based on 28 parameters of the first set of advertisements, such as the advertising interval, signal strength (e.g., voltage), etc.

In the example of FIG. 4, wake circuitry 138 may be configured to compare a voltage of each pulse of the set of pulses to a voltage threshold 150 and detect an advertising pattern based on the comparison of the voltage of each pulse of the set of pulses to voltage threshold 150. For instance, comparator 152 of wake circuitry 138 may include a first input 146, a second input 148, and an output 149. First input 146 may be configured to receive the set of pulses. Second input 148 may be configured to receive voltage threshold 150. Output 149 may be configured to output a signal based on a comparison of first input 146 and second input 148. For example, output 149 may output a ‘high’ signal when the voltage of a pulse is greater than the voltage threshold and output a ‘low’ signal when the voltage of a pulse is less than or equal to the voltage threshold. In this way, output 149 may output a modified set of pulses using the set of pulses from energy harvester module 140. In some examples, voltage threshold 150 may be pre-configured in software or hardware, determined by IMD 106, device 104, etc.

Voltage threshold 150 may be, e.g., pre-configured in software or hardware, determined by IMD 106, device 104, etc. Voltage threshold 150 may be set to control the sensitivity of wake circuitry 138. Voltage threshold 150 may be set based on the noise floor of system 100 and the required sensitivity. In some examples, voltage threshold 150 may be calculated for each patient depending on patient-based noise factors. For example, the noise floor may vary for each patient depending on factors including body temperature, a position of implant, other additional implants, etc. In some examples, a module configured to execute instructions from a computer-readable storage medium to output voltage threshold 150.

In some examples, wake circuitry 138 may include sensitivity circuitry 154. Sensitivity circuitry 154 may be configured to modify the amplitude of the output from output 149 from comparator 152. For example, sensitivity circuitry 154 may greatly decrease the amplitude of the output from output 149 such that the output is not detectable. As a result, wake circuitry 138 may not detect any advertising pattern, let alone an advertising pattern that can satisfy an advertising pattern requirement to activate telemetry circuitry 130. This may be useful in cases where it is highly improbably that telemetry circuitry 130 needs to be active (e.g., when IMD 106 is being shipped). Additionally, sensitivity circuitry 154 may maintain or even increase the amplitude of output 149 such that the output from output 149 is detectable (and therefore capable of being processed). IMD 106 may control sensitivity circuitry 154 based on user input.

Advertising pattern detection module 158 (e.g., a digital logic decoder sequencer) may receive the output from output 149 and determine whether an associated advertising pattern satisfies an advertising pattern requirement. For example, advertising pattern detection module 158 may determine whether the intervals between high signals match a predetermined advertising pattern. Responsive to the advertising pattern matching a predetermined advertising pattern, advertising pattern detection module 158 may determine that the advertising pattern satisfies the advertising pattern requirement. On the other hand, responsive to the advertising pattern not matching a predetermined advertising pattern, advertising pattern detection module 158 may determine that the advertising pattern of the set of pulses does not satisfy the advertising pattern requirement. Responsive to determining that the advertising pattern satisfies an advertising pattern requirement, advertising pattern detection module 158 may output an activation signal that causes telemetry circuitry 130 to transition from an inactive state to an active state.

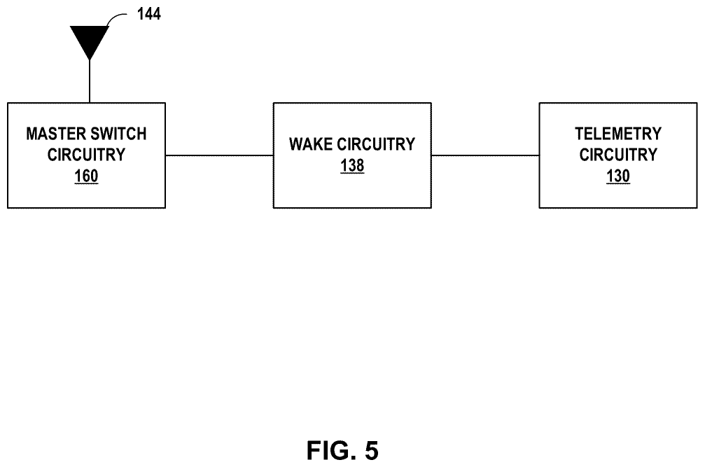

FIG. 5 is a block diagram of a portion of IMD 106. As shown in FIG. 5, IMD 106 may include antenna 144, wake circuitry 138, master switch circuitry 160, and telemetry circuitry 130. Master switch circuitry 160 may be configured to electrically connect and disconnect antenna 144 from wake circuitry 138. Master switch circuitry 160 may be distinct from switch circuitry 142. IMD 106 (e.g., processing circuitry 128) may control the state of master switch circuitry 160 (e.g., based on user input). Master switch circuitry 160 may electrically disconnect antenna 144 from wake circuitry 138. For example, IMD 106 may be preconfigured in a ‘shipping mode’ in which master switch circuitry 160 electrically disconnects antenna 144 from wake circuitry 138. The shipping mode may be useful when IMD 106 is being shipped or otherwise should not be communicating with another device. Then, a clinician or user (e.g., a patient or caretaker) may configure IMD 106 in an operating mode in which master switch circuitry 160 electrically connects antenna 144 to wake circuitry 138.

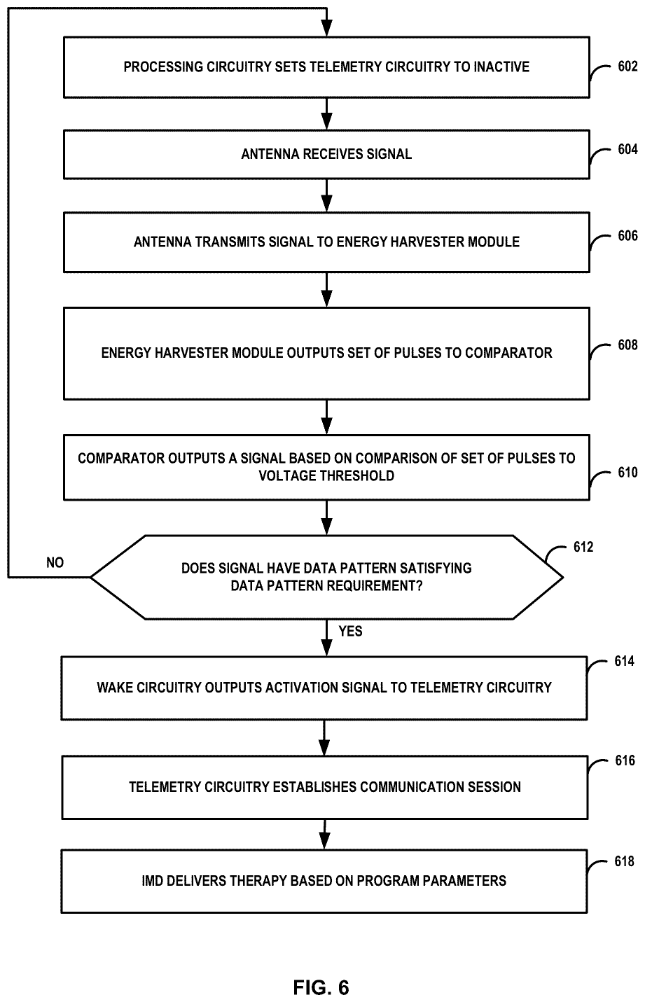

FIG. 6 is a flowchart illustrating an example operation of a medical device configured according to an example of the techniques of the disclosure. FIG. 6 is discussed with respect to FIGS. 1-5 for example purposes only.

Unless telemetry circuitry 130 is already inactive, processing circuitry 128 may set telemetry circuitry 130 of IMD 106 to inactive (602). When inactive, telemetry circuitry 130 may have reduced energy consumption. Antenna 144 may receive a signal carrying a first set of advertisements from device 104 (604). The first set of advertisements may be compliant with a communication protocol, such as BLE. For example, the signal carrying the first set of advertisements may be at a frequency band from 2.402 GHz to 2.48 GHz. Antenna 144 may transmit the signal to energy harvester module 140 (606). Responsive to receiving the signal, energy harvester module 140 may output a set of pulses based on the first set of advertisements to comparator 152 (608). In some examples, energy harvester module 140 may harvest energy from the signal carrying the first set of advertisements received by antenna 144.

Comparator 152 of wake circuitry 138 may receive the set of pulses via first input 146. Comparator 152 may compare first input 146 with second input 148 receiving voltage threshold 150 (610). For example, output 149 of comparator 152 may output a ‘high’ signal when the voltage of a pulse is greater than voltage threshold 150 and output a ‘low’ signal when the voltage of a pulse is less than or equal to voltage threshold 150. The voltage threshold may be, e.g., pre-configured in software or hardware, determined by IMD 106, device 104, etc.

Advertising pattern detection module 158 may receive the output from output 149 and determine whether an associated advertising pattern satisfies an advertising pattern requirement (612). For example, advertising pattern detection module 158 (e.g., a digital logic decoder sequencer) may determine whether the intervals between high signals match a predetermined advertising pattern. Responsive to the advertising pattern matching a predetermined advertising, advertising pattern detection module 158 may determine that the advertising pattern satisfies the advertising pattern requirement (“YES” branch of 612), and output an activation signal to telemetry circuitry 130 (614). The activation signal may cause telemetry circuitry 130 to activate and establish a communication session with device 104 (616) by outputting a second set of advertisements. The second set of advertisements may be compliant with the communication protocol of the first set of advertisements. The first set of advertisements and the second set of advertisements may be advertised on a set of frequency channels assigned for advertisements by the communication protocol of the first set of advertisements and the second set of advertisements.

On the other hand, responsive to determining that the advertising pattern does not match a predetermined advertising pattern the advertising pattern requirement, advertising pattern detection module 158 may determine that the advertising pattern does not satisfy the advertising pattern requirement (“NO” branch of 612), and not output anything such that telemetry circuit 130 remains inactive (602).

In some examples, telemetry circuitry 130 may receive program parameters via the communication session with device 104. The program parameters may represent values for various stimulation parameters such as magnitude and electrode combination. IMD 106 may deliver therapy based on the program parameters (618). For example, IMD 106 may deliver DBS, SCS, SNS, TDD, PNFS, TNS, pelvic stimulation, gastric stimulation, peripheral nerve field stimulation, etc., as discussed above.

The following examples are illustrative of the techniques described herein.

Example 1: A medical device configured to provide a therapy to a patient, the medical device includes wake circuitry configured to: receive a first set of data from a device associated with the medical device, wherein the first set of data is received at a frequency band; output a set of pulses based on the first set of data; detect a data pattern using the set of pulses; and responsive to a determination that the data pattern satisfies a data pattern requirement, output an activation signal; and the telemetry circuitry configured to: responsive to receiving the activation signal, output a second set of data, wherein the second set of data is transmitted at the frequency band; and establish a communication session with the device using the second set of data.

Example 2: The medical device of example 1, wherein the wake circuitry is further configured to: compare a voltage of each pulse of the set of pulses to a voltage threshold; and detect the data pattern based on the comparison of the voltage of each pulse of the set of pulses to the voltage threshold.

Example 3: The medical device of example 1 or 2, wherein the wake circuitry includes a comparator includes a first input configured to receive the set of pulses; a second input configured to receive a voltage threshold; and an output configured to output the data pattern.

Example 4: The medical device of any of examples 1 to 3, wherein the wake circuitry is configured to receive, by the telemetry circuitry and using the communication session, electrical stimulation information from the device associated with the medical device, and wherein the medical device is configured to provide the therapy based on the electrical stimulation information.

Example 5: The medical device of any of examples 1 to 4, wherein the second set of data includes a hop set of frequencies, and wherein the telemetry circuitry is configured to establish the communication session according to the hop set of frequencies.

Example 6: The medical device of any of examples 1 to 5, further including an antenna configured to operate at the frequency band.

Example 7: The medical device of example 6, further includes responsive to a determination that the data pattern satisfies the data pattern requirement, connect the telemetry circuitry and the antenna and disconnect the wake circuitry and the antenna; and responsive to a termination of the communication session, connect the wake circuitry and the antenna disconnect the telemetry circuitry and the antenna.

Example 8: The medical device of example 7, wherein, to determine whether the data pattern satisfies the data pattern requirement, the wake circuitry is configured to: determine one or more advertising intervals using the set of pulses, wherein the data pattern includes the one or more advertising intervals; responsive to the data pattern matching a predetermined advertising pattern, determine that the data pattern satisfies the data pattern requirement; and responsive to the data pattern not matching the predetermined advertising interval sequence, determine that the data pattern does not satisfy the data pattern requirement.

Example 9: The medical device of any of examples 1 to 8, wherein the device associated with the medical device includes one or more of an additional medical device, clinician programmer, a patient programmer, a recharger device, or a mobile device.

Example 10: The medical device of any of examples 1 to 9, wherein the first set of data is compliant with a communication protocol, wherein the second set of data is compliant with the communication protocol, and wherein the first set of data and the second set of data are transmitted on a set of frequency channels assigned for data transmissions by the communication protocol.

Example 11: The medical device of example 10, wherein the communication protocol is Bluetooth® low energy (BLE).

Example 12: The medical device of any of examples 1 to 11, wherein the wake circuitry is configured to harvest energy from a signal received by the wake circuitry, and wherein the wake circuitry is powered, at least in part, by the harvested energy.

Example 13: The medical device of any of examples 1 to 12, wherein the therapy includes one or more of deep brain stimulation (DBS), spinal cord stimulation (SCS), sacral neuromodulation (SNS), targeted drug delivery (TDD), pelvic stimulation, gastric stimulation, peripheral nerve field stimulation (PNFS), or tibial nerve stimulation (TNS).

Example 14: The medical device of any of examples 1 to 13, wherein the first set of data includes a first set of advertisements, wherein the second set of data includes a second set of advertisements, and wherein each advertisement of the first set of advertisements and the second set of advertisements includes information for connecting devices.

Example 15: A method includes receiving, by wake circuitry of a medical device configured to provide a therapy to a patient, a first set of data from a device associated with the medical device, wherein the first set of data is received at a frequency band; outputting, by the wake circuitry, a set of pulses based on the first set of data; detecting, by the wake circuitry, a data pattern using the set of pulses; responsive to a determination that the data pattern satisfies a data pattern requirement, outputting, by the wake circuitry, an activation signal; responsive to receiving, by telemetry circuitry of the medical device, the activation signal, outputting, by the telemetry circuitry, a second set of data, wherein the second set of data is transmitted at the frequency band; and establishing, by the telemetry circuitry, a communication session with the device using the second set of data.

Example 16: The method of example 15, further includes comparing, by the wake circuitry, a voltage of each pulse of the set of pulses to a voltage threshold; and detecting, by the wake circuitry, the data pattern based on the comparison of the voltage of each pulse of the set of pulses to the voltage threshold.

Example 17: The method of example 15 or 16, further includes receiving, by a first input of a comparator, the set of pulses; receiving, by a second input of the comparator, a voltage threshold; and outputting, by an output of the comparator, the data pattern.

Example 18: The method of any of examples 15 to 17, wherein the first set of data is compliant with a communication protocol, wherein the second set of data is compliant with the communication protocol, and wherein the first set of data and the second set of data are transmitted on a set of frequency channels assigned for data transmissions by the communication protocol.

Example 19: The method of any of examples 15 to 18, wherein the first set of data includes a first set of advertisements, wherein the second set of data includes a second set of advertisements, and wherein each advertisement of the first set of advertisements and the second set of advertisements includes information for connecting devices.

Example 20: A system includes a device associated with a medical device configured to provide a therapy to a patient, wherein the device includes one or more of an additional medical device, a clinician programmer, a patient programmer, a recharger device, or a mobile device; and the medical device includes wake circuitry configured to: receive a first set of data from the device, wherein the first set of data is received at a frequency band; output a set of pulses based on the first set of data; detect a data pattern using the set of pulses; and responsive to a determination that the data pattern satisfies a data pattern requirement, output an activation signal; and the telemetry circuitry configured to: responsive to receiving the activation signal, output a second set of data, wherein the second set of data is transmitted at the frequency band; and establish a communication session with the device using the second set of data.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A medical device configured to provide a therapy to a patient, the medical device comprising:
wake circuitry configured to:
receive a first set of data from a device associated with the medical device, wherein the first set of data is received at a frequency band;
output a set of pulses based on the first set of data;
detect a data pattern using the set of pulses; and
responsive to a determination that the data pattern satisfies a data pattern requirement, output an activation signal; and
the telemetry circuitry configured to:
responsive to receiving the activation signal, output a second set of data, wherein the second set of data is transmitted at the frequency band; and
establish a communication session with the device using the second set of data.

2. The medical device of claim 1, wherein the wake circuitry is further configured to:
compare a voltage of each pulse of the set of pulses to a voltage threshold; and
detect the data pattern based on the comparison of the voltage of each pulse of the set of pulses to the voltage threshold.

3. The medical device of claim 1, wherein the wake circuitry comprises a comparator comprising:
a first input configured to receive the set of pulses;
a second input configured to receive a voltage threshold; and
an output configured to output the data pattern.

4. The medical device of claim 1, wherein the wake circuitry is configured to receive, by the telemetry circuitry and using the communication session, electrical stimulation information from the device associated with the medical device, and wherein the medical device is configured to provide the therapy based on the electrical stimulation information.

5. The medical device of claim 1, wherein the second set of data comprises a hop set of frequencies, and wherein the telemetry circuitry is configured to establish the communication session according to the hop set of frequencies.

6. The medical device of claim 1, further comprising an antenna configured to operate at the frequency band.

7. The medical device of claim 6, further comprising switch circuitry configured to:
responsive to a determination that the data pattern satisfies the data pattern requirement, connect the telemetry circuitry and the antenna and disconnect the wake circuitry and the antenna; and
responsive to a termination of the communication session, connect the wake circuitry and the antenna disconnect the telemetry circuitry and the antenna.

8. The medical device of claim 7, wherein, to determine whether the data pattern satisfies the data pattern requirement, the wake circuitry is configured to:
determine one or more advertising intervals using the set of pulses, wherein the data pattern comprises the one or more advertising intervals;
responsive to the data pattern matching a predetermined advertising pattern, determine that the data pattern satisfies the data pattern requirement; and
responsive to the data pattern not matching the predetermined advertising interval sequence, determine that the data pattern does not satisfy the data pattern requirement.

9. The medical device of claim 1, wherein the device associated with the medical device comprises one or more of an additional medical device, clinician programmer, a patient programmer, a recharger device, or a mobile device.

10. The medical device of claim 1, wherein the first set of data is compliant with a communication protocol, wherein the second set of data is compliant with the communication protocol, and wherein the first set of data and the second set of data are transmitted on a set of frequency channels assigned for data transmissions by the communication protocol.

11. The medical device of claim 10, wherein the communication protocol is Bluetooth® low energy (BLE).

12. The medical device of claim 1, wherein the wake circuitry is configured to harvest energy from a signal received by the wake circuitry, and wherein the wake circuitry is powered, at least in part, by the harvested energy.

13. The medical device of claim 1, wherein the therapy comprises one or more of deep brain stimulation (DBS), spinal cord stimulation (SCS), sacral neuromodulation (SNS), targeted drug delivery (TDD), pelvic stimulation, gastric stimulation, peripheral nerve field stimulation (PNFS), or tibial nerve stimulation (TNS).

14. The medical device of claim 1, wherein the first set of data comprises a first set of advertisements, wherein the second set of data comprises a second set of advertisements, and wherein each advertisement of the first set of advertisements and the second set of advertisements comprises information for connecting devices.

15. A method comprising:
receiving, by wake circuitry of a medical device configured to provide a therapy to a patient, a first set of data from a device associated with the medical device, wherein the first set of data is received at a frequency band;
outputting, by the wake circuitry, a set of pulses based on the first set of data;
detecting, by the wake circuitry, a data pattern using the set of pulses;
responsive to a determination that the data pattern satisfies a data pattern requirement, outputting, by the wake circuitry, an activation signal;
responsive to receiving, by telemetry circuitry of the medical device, the activation signal, outputting, by the telemetry circuitry, a second set of data, wherein the second set of data is transmitted at the frequency band; and
establishing, by the telemetry circuitry, a communication session with the device using the second set of data.

16. The method of claim 15, further comprising:
comparing, by the wake circuitry, a voltage of each pulse of the set of pulses to a voltage threshold; and
detecting, by the wake circuitry, the data pattern based on the comparison of the voltage of each pulse of the set of pulses to the voltage threshold.

17. The method of claim 15, further comprising:
receiving, by a first input of a comparator, the set of pulses;
receiving, by a second input of the comparator, a voltage threshold; and
outputting, by an output of the comparator, the data pattern.

18. The method of claim 15, wherein the first set of data is compliant with a communication protocol, wherein the second set of data is compliant with the communication protocol, and wherein the first set of data and the second set of data are transmitted on a set of frequency channels assigned for data transmissions by the communication protocol.

19. The method of claim 15, wherein the first set of data comprises a first set of advertisements, wherein the second set of data comprises a second set of advertisements, and wherein each advertisement of the first set of advertisements and the second set of advertisements comprises information for connecting devices.

20. A system comprising:
a device associated with a medical device configured to provide a therapy to a patient, wherein the device comprises one or more of an additional medical device, a clinician programmer, a patient programmer, a recharger device, or a mobile device; and
the medical device comprising:
wake circuitry configured to:
receive a first set of data from the device, wherein the first set of data is received at a frequency band;
output a set of pulses based on the first set of data;
detect a data pattern using the set of pulses; and
responsive to a determination that the data pattern satisfies a data pattern requirement, output an activation signal; and
the telemetry circuitry configured to:
responsive to receiving the activation signal, output a second set of data, wherein the second set of data is transmitted at the frequency band; and
establish a communication session with the device using the second set of data.
